(19) United States
(12) Patent Application Publication
(10) Pub. No.: US 2005/0070897 A1
(43) Pub. Date: Mar. 31, 2005

(54) Laser triangulation of the femoral head for total knee arthroplasty alignment instruments and surgical method

(76) Inventor: Thomas D. Petersen, La Mesa, CA (US)

Correspondence Address: H. JAY SPIEGEL, P.O. BOX 11, Mount Vernon, VA, 22121, US

(21) Appl. No.: 10/671,554

(22) Filed: Sep. 29, 2003

(51) Int. Cl. A61F 5/04

(52) U.S. Cl. 606/53; 600/102

(57) ABSTRACT

An Extramedullary system of alignment for total knee arthroplasties uses a small diode laser at the center of the knee adjustable to the longitudinal axis of the femur to triangulate the center of the femoral head. It utilizes a V-Frame positioning device that fits into the distal femoral intercondylar notch and is tangent to the articular surfaces of the notch. It is also parallel to the anterior femoral cortex by using a removal tongue flange that sits flat on the filed surface of the anterior cortex. This prepositions the Distal Femoral Resector Guide within a few degrees of the center of the femoral head. An adjustment knob on the V-Frame pivots the distal femoral resector guide to the exact center of the femoral head for that particular patient accomplishing fine adjustment of the longitudinal axis of the femur. There is only one position where the laser beam will go through the center of the target no matter where you position the leg and that is when the target's bulls-eye is exactly over the rotational center of the femoral head. Since the laser confirms this position, the surgeon is assured that the alignment is accurate. The Distal Femoral Resector Guide is then fixed to bone with fixation pins and the resection made with a power saw. The laser is moved to the target mount to act as a longitudinal “laser ruler” for the remainder of the operation.

### BACKGROUND OF THE INVENTION

The present invention relates to laser triangulation of the femoral head for total knee arthroplasty alignment instruments and surgical method. Orthopedic surgeons have been struggling with the alignment of total knee arthroplasties since their inception in the early 1970s. Basically, what is necessary is a 5-7 degree angular resection of the distal femoral condyles as related to the mechanical axis of the femur and a perpendicular resection of the proximal tibia as related to its central axis. The mechanical axis is defined as a line extending from the center of the femoral head through the center of the knee to the center of the ankle. Early on, resections of the distal femur and proximal tibia were made by visually trying to match the existing anatomy by eye. Alignment varied considerably depending on the skill of the operating surgeon.

In the early 1980s, precision jigs were introduced that aligned the resections to the mechanical axis of the respective bones. Initially, these were extramedullary jigs, where the alignment was done outside of the bone by extended rods with perpendicular cutting heads. Later, the intramedullary rod method became popular. This technique required the rod to extend through the intramedullary canal of the patient's femur and tibia.

Many sophisticated instrument systems have emerged over the last 25 years to make these difficult resections more reliable and reproducible for the average orthopedist. Approximately 250,000 total knee arthroplasties are done in the USA each year. After 35 years, there is a wealth of knowledge of the failure modes of this procedure. The most important parameter is accurate alignment of the components. It has been proven that only 4.5 degrees of misalignment causes the components to only load one side of the knee joint leading to rapid failure of the implant. The literature strongly supports the conclusion that the closer the surgeons approach neutral alignment, as defined as equal weight bearing on both sides of the joint, the more successful the implant system will be with longevity, so far, up to 30 years in some reported series. Misaligned total knee arthroplasties tend to get worse with time because the abnormal weight distribution accelerates the wear on the overloaded side leading to rapid failure within a few years in the case of the grossly malaligned.

Although total knee alignment is made up by two resections, one on the distal femur and one on the proximal tibia, the proximal tibia resection is relatively easy because the landmarks are visible to the surgeon. Consequently, this discussion will be limited to the distal femoral cut, which is blind to the surgeon because the femoral head location cannot easily be determined.

There are basically two standard alignment methods for total knee surgeries. The first, as explained above, is called the extramedullary method meaning the alignment is accomplished without inserting a rod into the intramedullary canal of bone. This method requires the femoral head to be located either by x-ray or the surgeon's educated guess. Then an extended rod with a perpendicular resection guide is used to make the cuts on the distal femoral condyles by holding one end of the rod at the center of the femoral head and the perpendicular resection guide at the level of resection at the center of the knee.

Two patented systems known to Applicant disclose a variation of this extramedullary method. The first is a patent by Dance et al. (U.S. Pat. No. 5,690,638) called “A Method and Apparatus for the Alignment of a Femoral Knee Prosthesis.” This method uses a pivotable resection guide at the distal condyles that allows a distal retraction force in a freely suspended knee that locates the center of rotation of the femoral head. The resection guide is then locked into position and the cuts made on the distal femur.

There are three problems with this system. The first is that it requires a rigid fixation system of the resection head to the distal femur because a substantial force is necessary to rotate the cutting head to the center of rotation. Their described attachment system requires extensive resection of soft tissues from the distal femur. The second problem is that the system requires the patient's entire leg be suspended freely so no external restrictions will affect the subsequent traction procedure. The elaborate suspension system is expensive and time consuming to set up. The third problem is that the surgeon has no way to check the accuracy of the traction procedure and has to proceed on faith that the system is accurate.

Another variation of the extramedullary method is disclosed in a patent disclosing an invention for which the applicant herein, Thomas D. Petersen, is a co-inventor, (U.S. Pat. No. 5,606,590), titled “A Surgical Laser Beam-Based Alignment System and Method.” This patent is an improvement over the extramedullary alignment assist device disclosed in U.S. Pat. No. 4,524,766, “A Surgical Knee Alignment Method and System.” In this prior patent, the use of radiographically opaque L arm located over the patient's femoral head was described.

Applicant's co-invention (U.S. Pat. No. 5,606,590) utilizes a laser and a sophisticated X-ray cassette with a radiograph scale above and below the patient's hip to enable the surgeon to minimize parallax error. If the X-ray beam is not exactly perpendicular to the patient's hip, there is parallax error showing up on the X-ray as to the location of the femoral head. A laser is then set up in line with the center of the patient's femoral head and used to fix the distal femoral resector perpendicular to this longitudinal axis.

The problems with this system include the elaborate X-ray system that requires proper positioning of the patient on the custom X-ray cassette and the time it takes to set up, take and interpret the X-ray. Then the laser needs to be adjusted to the center of the femoral head and calibrated to the longitudinal axis. Although everything has been done to eliminate parallax error, the patient's femur is only roughly equidistant between the radiographic scales so there is still residual inherent error present in the system, albeit only more than one degree in 10 percent of patients.

The other, more commonly used, method of alignment for total knee arthroplasties, as explained above, is the intramedullary method. This method uses a rod placed through a drill hole in the notch between the distal femoral condyles into the intramedullary canal of the long axis of the femur. The distal end of the rod has a protractor adjustment that allows the surgeon to dial in an angulated resection of the distal femoral condyles, typically 5-7 degrees laterally off the central axis of the rod.

There are three major problems with intramedullary alignment systems. The first is an inherent spatial mechanical error of up to 2-3 degrees in both saggital and axial alignment because the intramedullary canal has a cross-section about the size of a nickel. Consequently, the rod can be angulated within that space in both planes, worse case, 2-3 degrees, especially in larger femurs.

The second major problem is, not infrequently, the femur is deformed so that it cannot provide a reliable guide. Congenital bowing seen in dwarfism such as Morguio's disease and bone dysplacias are examples where this system should not be used. Previous trauma with resultant angulations of the canal is another example where the intramedullary system should not be used because of significant error.

Lastly, there are well-documented medical complications when the intramedullary system is used that are not present with the extramedullary approach. The medical literature is full of articles that document the increased risk from fat embolism and increased blood loss with the intramedullary method. Usually, there is 50 percent more blood loss when the intramedullary canal is used for alignment. Patients require more blood transfusions with this method and accept the risks that go with blood transfusions.

Fat embolism can be a serious problem. The intramedullary fat, dislodged by the drilling and placement of the rod, gets into the patient's blood stream and in most cases causes lethargy and sensorial changes that inhibit the patient's post-operation rehabilitation and delay hospital discharge at least one day.

With these problems in mind, the present invention was developed to improve alignment for total knee arthroplasties and subsequent increased longevity of the implant system while minimizing the intrusiveness of the instruments.

### SUMMARY OF THE INVENTION

The present invention relates to laser triangulation of the femoral head for total knee arthroplasty alignment instruments and surgical method. The present invention consists of a newly developed extramedullary method to align the Distal Femoral Resection for Total Knee arthroplasty without violating the intramedullary canal. This method preferably uses a miniature high tech laser to triangulate the center of the femoral head without the need for an inter-operative X-ray.

By avoiding IM rods for alignment, considerable morbidity will be spared. In studies by Applicant using Applicant's Laserlign I system, covered by U.S. Pat. No. 5,606,590, an extramedullary alignment system in over 500 cases (several surgeons over the past 5 years) has shown marked improvement in the following: 1) 50% lower blood loss rarely requiring transfusion; 2) marked improvement in the patient's sensorium and lethargy post surgery due to subacute fat embolism, well documented in several studies, allowing the patients to be discharged from the hospital, on the average, one day earlier; 3) improved alignment greater than 90% within 1.5 degrees of optimal alignment.

Surgeons who have used the Laserlign I system appreciate the Laser Ruler that can be used throughout the case to confirm the Mechanical Axis allowing for inter-operative adjustments.

The present invention, known as the Laserlign II, is actually more accurate than the Laserlign I system that required an X-ray to determine the center of the femoral head. It is intuitive and based on sound mechanical principles that leave no doubt the analysis is correct. The laser gives the surgeon visual confirmation compared to other extramedullary systems that are based on faith that the mechanical principle is correct.

The Laserlign II system can be used with any commercial Total Knee System. The Laser keys up on the Distal Femoral Resector, therefore this vital instrument is included in the Laserlign II system. Since the Laser/Resector must be as flat as possible to the anterior femoral cortex, several other instruments that prepare the distal femur for the Laser/Resector are included as well. The other commercial systems pick up after the preliminary cut on the anterior femoral condyles and the Laserlign II resection of the distal femoral condyles.

The procedure includes a Laser that is set up over the femoral head on a platform that creates a Laser Ruler, i.e., the Mechanical Axis, for the surgeons to refer to throughout the case to make sure their alignment is correct. It is extremely gratifying to be able to confirm on the operating table that the alignment is correct.

The present invention utilizes a small diode laser positioned at the center of the patient's knee that is adjustable to the longitudinal axis of the femur to facilitate triangulation of the center of the patient's femoral head.

The present invention includes the following interrelated objects, aspects and features:

(1) The present invention includes a removable diode laser that attaches to the distal femoral resector guide at the knee. A V-Frame positioning instrument is provided that has a central pivot allowing adjustment of the longitudinal axis of extension of the laser beam about this central pivot.

(2) The distal femoral resector guide is attached by a pivot post on the V-Frame, and a removable retractor shield is attached thereto.

(3) A clamping mechanism is provided to facilitate clamping of a laser target in a suspended location above the surgical table. In this regard, a clamping device clamps a vertical attachment arm to the table and a horizontal bar supports the laser target.

(4) The method of performing surgery in accordance with the teachings of the present invention is disclosed in detail. After the laser target has been positioned over the hip, an estimate is made of the location of the femoral head. Thereafter, the distal femur is prepared including preparation of a centralized intercondylar notch and a flat plane on the anterior femoral cortex using round and flat rasps, respectively. Thereafter, the top portions of the anterior femoral condyles are removed using a resector guide for that purpose.

(5) The inventive V-Frame is attached to the distal femur and a femoral resector guide is attached to the V-Frame. With this instrumentation in place, the next step is to mount the laser beam generator in a tapered slot on the distal femoral resector guide.

(6) Surgical techniques to be described in greater detail hereinafter facilitate location of the center of the femoral head using the laser beam on the target that had previously been located in an estimated location with regard to the femoral head. When these adjustments are completed, the bulls-eye of the laser target is now positioned directly over the center of the femoral head and the laser beam extends precisely down the mechanical axis of the leg.

(7) With these steps being accomplished, accurate resection of the distal femoral condyles may be performed to facilitate completion of total knee surgery.

As such, it is a first object of the present invention to provide laser triangulation of the femoral head for total knee arthroplasty alignment instruments and surgical method.

It is a further object of the present invention to provide such an apparatus and method in which a target is positioned over the hip of the patient.

It is a still further object of the present invention to provide such a device, which allows interconnection with a distal femoral resector guide.

It is a yet further object of the present invention to provide such a device in which a laser beam is used to dramatically enhance the accuracy of the resections that must be carried out during the performance of total knee surgery.

These and other objects, aspects and features of the present invention will be better understood from the following detailed description of the preferred embodiment when read in conjunction with the appended drawing figures.

### BRIEF DESCRIPTION OF THE DRAWINGS

FIG. 1 shows a side perspective view of a surgical table with a patient thereon and a target positioner mounted on the surgical table.

FIG. 2 shows a further side perspective view rotated from the view of FIG. 1, partially exploded, and showing the laser target mounted on the attachment arm.

FIG. 3 shows a further side perspective view depicting the manners of adjustment of the laser target with respect to the hip of the patient.

FIG. 4 shows a perspective view of a distal femur showing the position of a rasp used to create a centralized intercondylar notch.

FIG. 5 shows three separate views of the distal femur showing the use of a flat rasp to create a flat plane on the anterior femoral cortex of the distal femur.

FIG. 6 shows the use of an anterior condyle resector guide to resect a planar surface on the top of the distal femur by resecting portions of the anterior femoral condyles.

FIG. 7 shows a front perspective view of the distal femur showing the mounting of a V-Frame thereon.

FIG. 8 shows a side perspective view of the distal femur with a distal femoral resector guide mounted on the V-Frame.

FIG. 9a shows an exploded side view of a laser device.

FIG. 9b shows a cut away-section of the laser device of FIG. 9a.

FIG. 10 shows a side perspective view of the laser device mounted on the distal femur via the distal femoral resector guide.

FIG. 11 shows use of the laser to align a planar laser beam with the target mounted over the hip of the patient.

FIGS. 12 and 13 show a front perspective view of the Target illustrating the geometry of alignment of the knee laser beam to the center of the femoral head.

FIG. 14 is a blown-up of the front perspective view that illustrates correction of the knee laser beam to the center of the femoral head.

FIG. 15 shows a front perspective view of an adjustment knob used to align the knee laser beam.

FIG. 16 shows a front perspective view of the laser target when the target is correctly placed over the center of rotation of the femoral head.

FIG. 17 shows a side perspective view of the laser beam properly aligned along the mechanical axis of the patient's leg.

### SPECIFIC DESCRIPTION OF THE PREFERRED EMBODIMENT

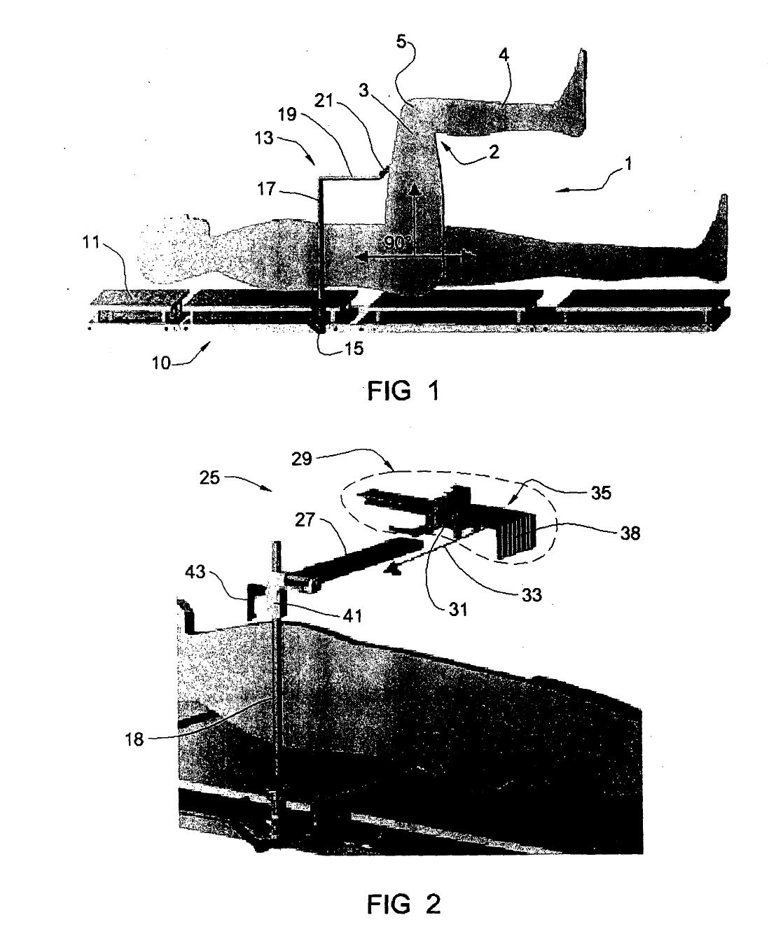

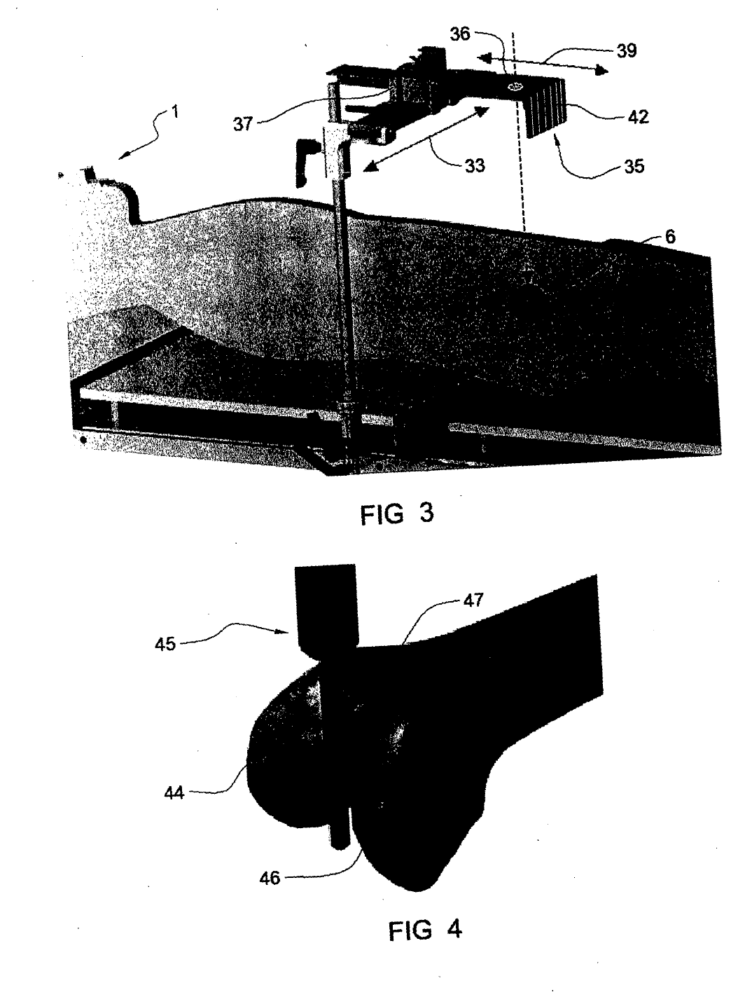

Reference is first made to FIGS. 1-3, which depict the initial positioning of a target over the hip of the patient. The patient is designated by the reference numeral 1 and has a leg 2 including an upper leg 3, a lower leg 4, and a knee 5, as well as a hip 6 (FIG. 3). A surgical table 10 has a surface 11 on which the patient 1 is positioned. A non-sterile target positioner 13 includes a non-sterile clamp 15 that is suitably clamped to the table 10, a vertical portion 17, and a horizontal portion 19 from which extends a perpendicular portion 21 designed to engage the patient's thigh when the upper leg 3 is flexed 90 degrees with respect to the patient's body (FIG. 1) and the surgical table, to position the patient on the operating table. Once the patient is properly positioned, the target positioner is removed from the clamp 15, whereupon the laser target system 25 is mounted on the clamp 15 (FIGS. 2-3).

As particularly seen in FIGS. 2 and 3, the laser target system 25 includes a bracket consisting of an attachment arm 18 sized to be received by the clamp 15 and a horizontal slide 27 on which the target portion 29 with a complementary recess 31 is mounted. As seen in FIGS. 2 and 3, the interaction between the horizontal slide 27 and the recess 31 allows sliding movements in the direction of arrow 33 (FIGS. 2 and 3). Additionally, the target portion 35 is mounted on a further slide mechanism 37 allowing reciprocation of the target 35 in the direction of the arrow 39 (FIG. 3).

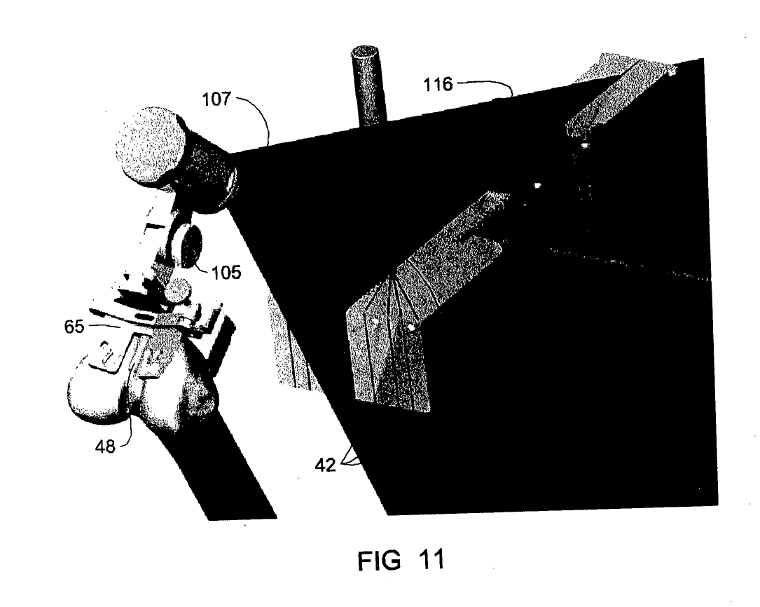

The vertical support 18 has the horizontal slide 27 mounted thereon using a mount 41 that has an opening through which the vertical support 18 extends and a screw clamp 43 that allows fixing the vertical position of the mount 41 in any desired vertical position. Thus, through use of the adjusting means described above, the target portion 29 may be adjusted in position in three degrees of freedom, all the movements being perpendicular to each other, taking into account all the adjustments described above, to allow the target portion 29 to be positioned over the hip 6 of the patient 1 so that, during the course of practice of the inventive surgical procedure, a laser beam 107 (FIG. 11) can be aligned over the target 35 while also extending precisely along the mechanical axis of the patient's leg. As seen in FIGS. 2-3, the target portion 29 has a longitudinal arm comprising a bulls-eye 36 on its top surface and a front surface comprising a vertically depending surface 38 having a plurality of vertical lines 42 (FIGS. 3 and 11) provided to facilitate alignment of the laser.

With reference now to FIGS. 4-6, an explanation will be made of the steps that are taken in practicing the inventive method to prepare the distal femur for attachment of a V-Frame and a distal femoral resector guide.

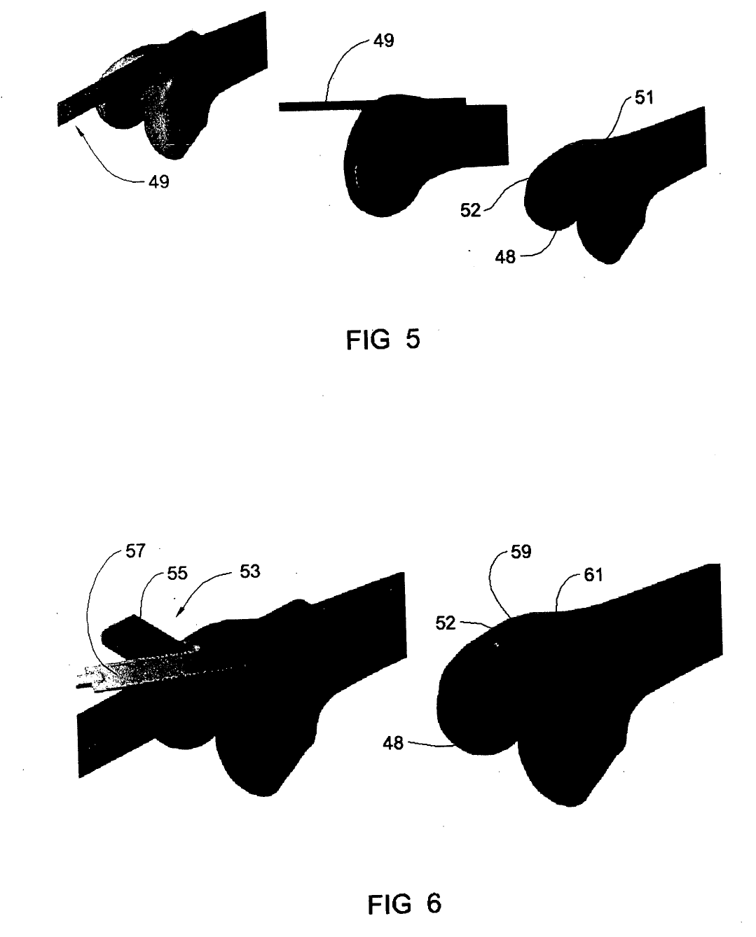

As shown in FIG. 4, a rasp 45 having a round cross-section is employed to centralize the intercondylar notch 47 located between the condyles 44 and 46 forming the filed notch 48 (FIG. 5). Thereafter, with reference to FIG. 5, a flat rasp 49 is used to create a flat plane 52 on the anterior femoral cortex 51 of the distal femur. Thereafter, with reference to FIG. 6, an anterior condyle resector guide 53 is placed on the planar surface 52 formed by the rasp 49, with the guide 53 having a flat guiding surface 55. A surgical saw 57 is employed to resect the top portion of the anterior femoral condyles as shown in the right-hand view of FIG. 6, to form a planar surface 59, and a planar surface 61, with the surfaces 59 and 61 being ⅛″ higher than the surface 52 but parallel with the surface 52 formed by the rasp 49. This is seen in FIG. 6.

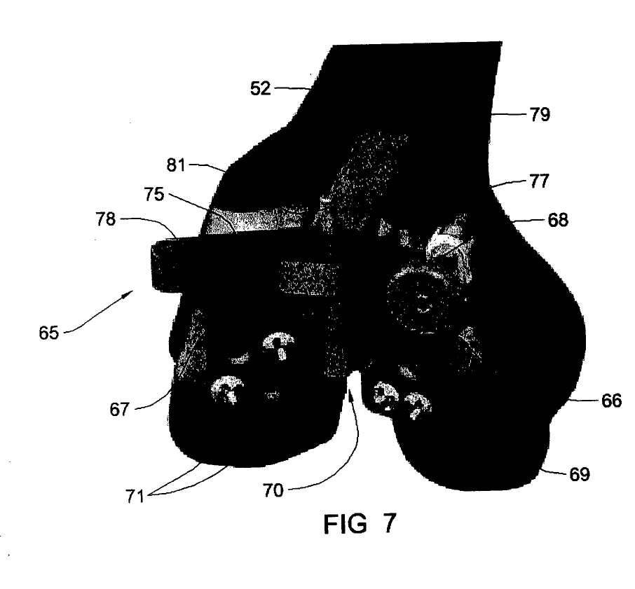

With reference to FIG. 7, a V-Frame 65 includes a downwardly depending pair of angled brackets 67 and 69 that are attached to the distal end of the femur using cortical screws 71. The V-Frame 65 includes angled wings 75 and 77, and a removable tongue 79 extends proximally toward the hip lying on the plane formed by the surface 52 (see FIG. 5).

The V-Frame 65 also includes an upwardly extending post 81 at the apex between the wings 75 and 77. Wing 75 has engraved rotational indicia 78 on its top surface 78. Extending downward is the notch guide 70 that fits into the filed notch or recess 48 (FIG. 5).

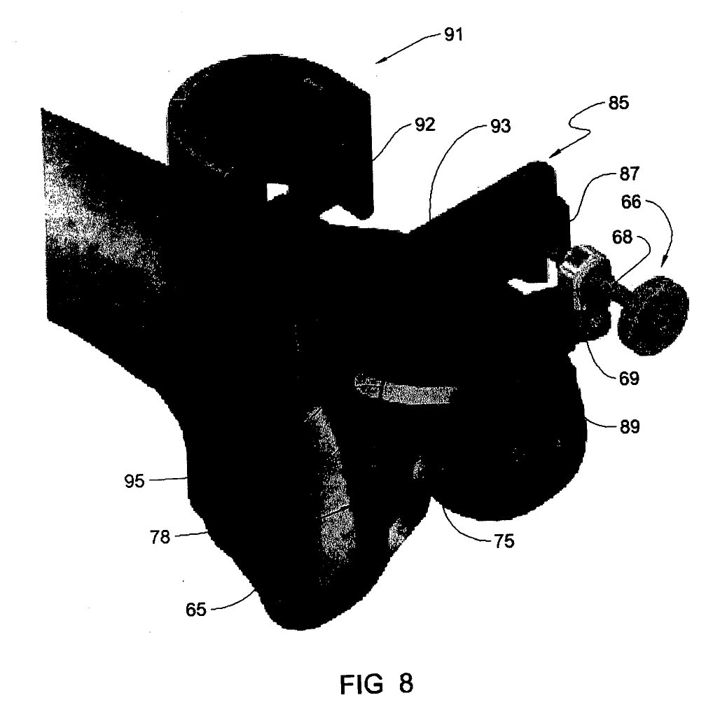

With reference now to FIG. 8, a distal femoral resector guide 85 is mounted on the V-Frame 65 with an underside recess hole (not shown) fitting over the post 81 and with the adjustment knob 66 being rotated to adjust the stem 68 through the rotatable stem housing 69 with respect to a recess 87 in the guide 85. The guide includes a first mount consisting of tapered laser slot 89 as well as a retractor shield 91, that locks into an “L” slot 92 and a cutting slot 93 provided to facilitate resection of the distal femur. An angular indicator 95 is also provided on the guide 85 that correlates to engraved indicia on the top surface 78. Rotation of knob 66 results in rotation of guide 85 about post 81 to adjust the relative position of guide 85 with respect to V-Frame. The indicator 95 is positioned with respect to the rotational indicia 78 on wing 75 to indicate the relative position.

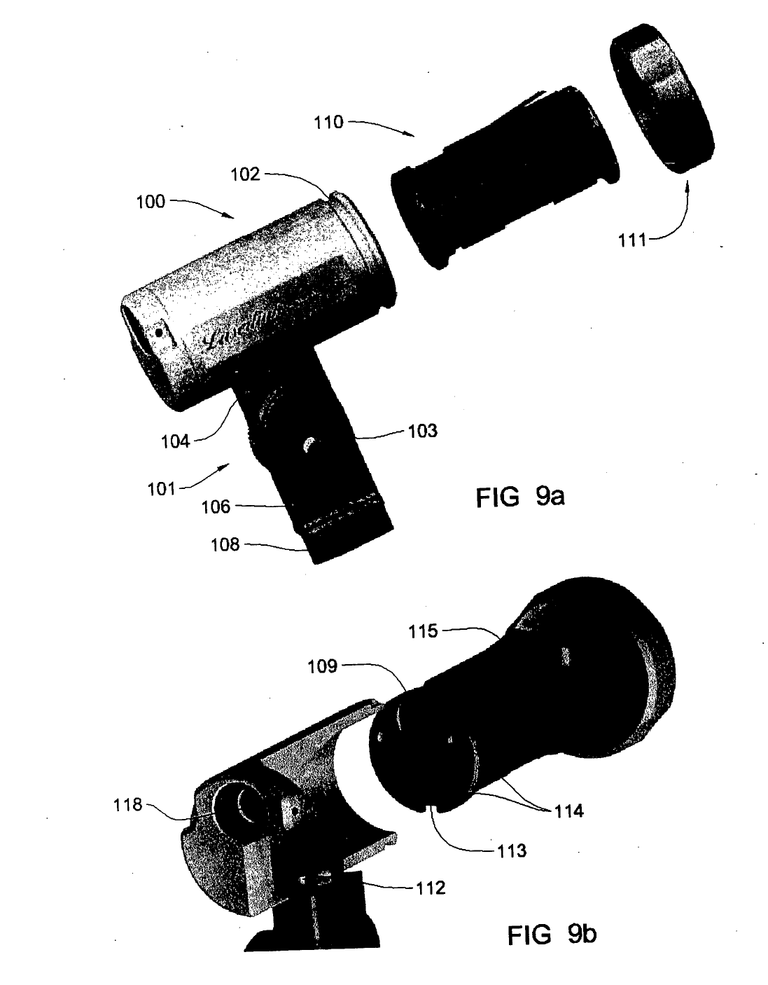

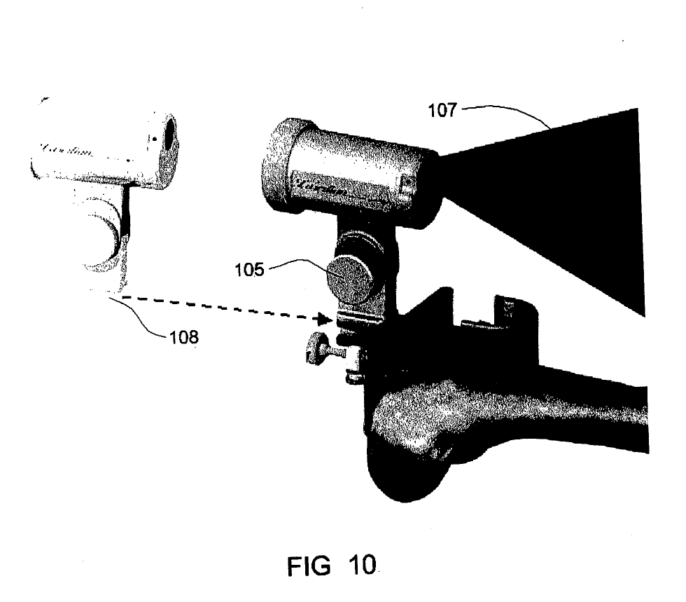

With reference now to FIGS. 9a and 9b, it is seen that a sterile laser canister 100 includes a bracket or stem 101 that is designed to be inserted into the laser slot 89 as seen in FIG. 10 with the tapered body or mount 108 under the slot 89. The stem 101 includes a swivel 103 that includes an adjustment knob 105 (FIG. 10) that may be loosened to allow pivoting of the halves 104, 106 of the stem 101 about the pivot 103, whereupon the knob 105 may be tightened to lock the angular relation between the halves 104 and 106. Manipulations of the adjustment knob 66 enable one to adjust the angular position of the saw guide slot 93 as well as of the laser slot 89 with the canister 100 following along as it is mounted within the laser slot 89. Thus, the canister 100 can have its position adjusted up and down and side to side with respect to the distal femur to facilitate aiming at the target 35 (see FIG. 3).

A laser module 110 is snugly mounted within the laser canister 100 (FIG. 10) and a sterile cap 111 is fastened over the opening 102 of the canister 100 to enclose the laser module therein. In the preferred embodiment of the present invention, the laser module 110 consists of a diode laser capable of emitting a highly linear beam of light, the direction of which may be adjusted in the manner explained above. Alignment is maintained between the diode laser and the canister by interengaging structures consisting of pin 112 entering into slot 113 and recesses 114 entering into pegs on the back surface of the lens face 118 module 110 (not shown). The canister 100 maintains proper alignment of the module 110 via the tapered body 108 located under the slot 89 (FIGS. 8 and 10). The laser module 110 has a diode laser generator 109 and an on-off switch 115 on its superior surface that turns on when it is inserted into the canister and turns off when it is removed (FIG. 9b).

With the apparatus of the present invention having been described in detail, the surgical method employing this apparatus will now be described in detail.

Step 1: The Setup

A) Position the Patient on the Operative Table:

Apply the non-sterile Operative Table Clamp 15 to the side rail of the operative table 10 as far distally as it will go in the second section of the operative table. Be sure this is on the same side as the operative knee 5. As the patient is being positioned on the operative table, use the non-sterile Target Positioner 13 within the Operative Table Clamp 15 so that when the patient's hip is flexed to 90 degrees (FIG. 1), it just touches the horizontal arm 21 of the Positioner. Move the patient up or down, or if the OR table clamp can be move cephalad without moving the patient do so. This will ensure that the Target will be correctly positioned over the patient's hip (FIG. 3). Now prep the knee and drape in the usual fashion.

B) Apply the Laser Target Over the Hip:

Apply the sterile Target attachment arm 18 to the operative table through a small slit through the drapes into the operative table clamp 15. Cover the slit in the drapes with a towel and tightly towel clip. Hold the Attachment Arm 18 rigid with the adjustment holes pointing to the patient's head while the nurse tightens the Attachment Arm 18 into place into the operative table clamp. Now apply the Horizontal Bar 27 pointing distally to the Attachment Arm. Be sure the arm is flipped over to the correct side and tighten the adjustment screw 43 through mount 41 when the arm is about 6 inches above the patient's abdomen (you need the height to flex the knee). Now slide the Laser Target system 29 on the Horizontal Bar from the open end. Be sure the center of the target portion 38 is facing distally. The actual center of the femoral head will be distal to the Hip Target leading edge by the bulk of the soft tissues on the anterior thigh.

C) ESTIMATE LOCATION OF THE FEMORAL HEAD

Adjust the Target's bulls-eye 36 (FIG. 3) transversely 33 to be centered over the femoral head 6, approximately 2 inches medially to the anterior superior iliac spine (ASIS) (The lateral edge of the target will be directly over the ASIS). Pull the Laser Target distally 39 about 2.5 inches from the closed position. The center of the target (bulls-eye 36) will now be over the approximate center of the femoral head. It is vitally important that the leading edge of the target be pulled past the center of the femoral head by at least 2 inches to facilitate the triangulation process. If there is not enough adjustment in the target, move the operative table clamp 15 distally. This moves the entire target distally, resulting in more adjustability.

For the triangulation process to work, you will need to be able to move the operative leg at least 10 to 15 degrees (in neutral rotation) in both adduction and abduction. In the rare case of ankylosis or significant contracture or the hip, another method of alignment must be used because the center of rotation will not be obtainable by laser triangulation. Using the standard approach to total knee arthroplasty, direct your attention to the distal femur.

Step 2: Prepare the Distal Femur

A) Centralize the Intracondylar Notch

File the intracondylar notch with a 5/16-inch (0.312) Rasp 45 (FIG. 4) to form filed notch 48 (FIG. 5). It is a vital importance to determine the exact center of the knee which, is usually, located slightly medial to the existing notch. Remove all the osteophytes and deepen the notch 47 with the rasp. It is important that the V-Frame 65 bottoms out on the adjacent condyles and is not held proud by the notch guide 70.

B) Create Flat Plane on Anterior Femoral Cortex

File the anterior intercondylar notch 51 flush with the anterior femoral cortex with a ⅝ inch flat Rasp 49 to form flat surface 52 (FIG. 5). Aim 5-7 degrees medially, which is the direction of the femoral head. There is usually a 5-10 degree medial slope of the anterior femoral cortex so the file will take more bone off on the lateral side. The cutting guide will sit on this surface so make it as flat as possible.

C) Remove Anterior Femoral Condyles

Use the Anterior Condyle Resector Guide 53 (FIG. 6) flush on the filed plane 52 in the intercondlylar notch to resect the anterior femoral condyles 59 & 61 with the saw 57 flush with the top 55 of the guide 53. This leaves ⅛ inch of anterior condylar bone above the filed central plane 52.

Step 3: Apply Hardware to Distal Femur

A) Apply V-Frame to Distal Femur

Apply the V-Frame 65 with the Tongue 79 flush to the filed anterior femoral cortex 52 (FIG. 7). Hold the ¼ inch Notch Rod 70 tight into the intercondylar notch recess 48 making sure the distal femoral condyles engage the V-Frame 65 brackets 67 & 69. Pre-drill the outer cortexes only, then screw cortical screws 71 into at least two holes on each side. Make sure the Tongue 79 is still flush with the anterior femoral cortex 52 while affixing the V-Frame 65 to bone.

B) Apply Distal Femoral Resector to V-Frame

NOTE: Remove the Tongue 79 before proceeding with this next step. Failure to do so will impede the resection of the distal femoral condyles.

Apply the Distal Femoral Resector Guide 85 to the V-Frame 65 (FIG. 8) and, at the same time, center the Adjustment knob stem 68 so it fits into its receptor 87 on the resector guide 85. This locks the resector guide into rough alignment with the center of the femoral head. Be sure the resector is set to the 90-degree mark 95 that corresponds to the 90-degree mark of the indicia 78 on the superior surface of the V-Frame 65 (the resector indicator arm will be flush with the anterior edge of the V-Frame). Next apply the Retractor Shield 91 into the square slot 92 at the proximal end of the Distal Femoral Resector Guide 85 (FIG. 8) and lock into place by pushing down and distally. This retractor shield will allow good visibility of the fixation holes on the proximal arm of the Distal Femoral Resector Guide.

Step 4: Laser Setup & Alignment of The Laser

The Laser Module 110 (FIGS. 9a & 9b) used in this procedure cannot be sterilized! Steam sterilization would damage the delicate laser components. Therefore, the nurse inserts the non-sterile laser module 110 into the Sterile Laser Canister 100. The groove 113 on the laser module must be aligned with the rib 112 on the laser canister. The laser module lines up with the lens 118 of the canister. The laser automatically illuminates when inserted, and turns off when removed by the switch 115. The surgeon closes the canister with the sterile cap 111. The surgeon then inserts the self-centering alignment flange 108 on the bottom of the canister into the dovetailed alignment groove 89 on the Distal Femoral Resector Guide 85 (FIG. 10). The same Laser will later be moved to the Target mount 116 (FIG. 11) for creating the Mechanical Longitudinal Axis 140 (FIG. 17) to be used to check alignment throughout the case.

The Laser Module 110 is now locked into alignment with the Distal Femoral Resector Guide 85 (FIG. 10) and is located at the midpoint of the knee and emits an angulated laser beam 107. What needs to be determined now is the center of the femoral head, so the Mechanical Axis of the Femur can be located accurately.

Alignment of the Laser

With reference to FIG. 11, first apply the Laser at the Knee; line up the knee laser beam 107 to be co-linear by positioning the leg, and moving the Target in the transverse plane 33 (FIG. 3) until the laser beam illuminates the centerline 117 of the target 35. Be sure to hold the leg as straight as possible, parallel with the operating table. It is also very important to keep the laser line beam 107 positioned vertically, keeping the beam parallel with the vertical markers 42 on the Target. Rotation of the knee will impair the accuracy of the laser beam. Therefore, it is important to always make sure the beam is parallel to the vertical lines on the front of the Target Vertical Adjustment 38 whenever making a determination.

A word of caution before starting the triangulating process: Occasionally, the laser beam will not illuminate the top of the target. This is likely in a patient with a fat thigh. In this case, use the swivel mechanism 103 on the side of the LASER to tilt the Laser beam upward until it shows on the top of the target. Also, the entire target can be moved up for a flexed thigh hitting the underside of the target. Two people are needed, one to hold the leg parallel to the lines on the Target and the other to make the target adjustments.

Step 5: Locating the Femoral Head

First: Determine the Longitudinal Axis of the Knee Laser

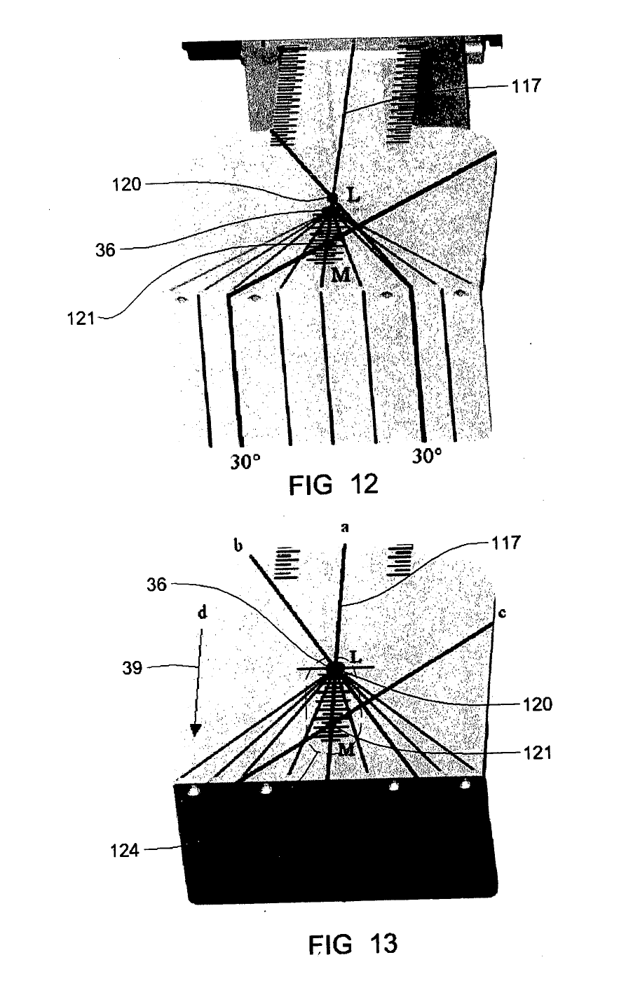

a. Line up the Knee Laser Beam 107 with the centerline 117 on the Target 35 (FIG. 11).
This preliminary longitudinal axis will be within a few degrees of the actual longitudinal axis because of the anatomical fit of V-Frame 65 in the femoral intercondylar notch 48 and the tangential surfaces of the distal femoral condyles (FIG. 11).
b. Swing the patient's flexed leg laterally (L) to 30 degrees and hold in place. Make a small dot on the target centerline 117 where it intersects the laser line beam 107. Label this dot L 120 (FIG. 12).
c. Next, sweep the leg medially (M) to 30 degrees and hold in place. Make another small dot on the target centerline 117 at the laser line beam 107 intersection. Label this dot M 121. NOTE: If the points are coincident, skip ahead to Step 2 (FIG. 12).
d. Move the Target longitudinally 39 so the bulls-eye 36 is on the centerline of the more superior dot of the two beams, 120 for this illustration (FIG. 13).

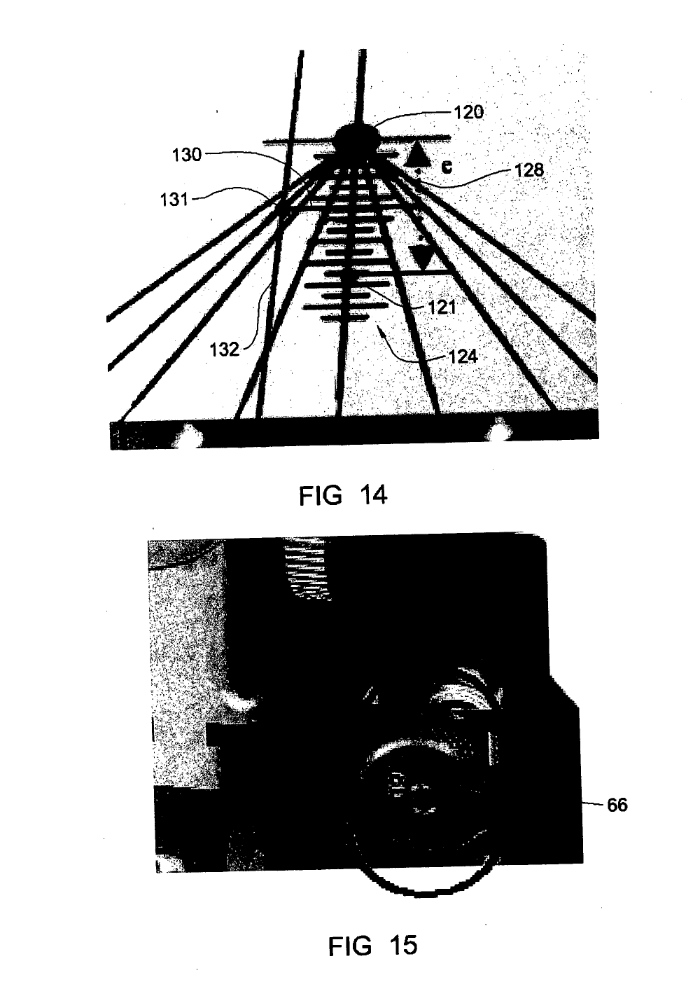

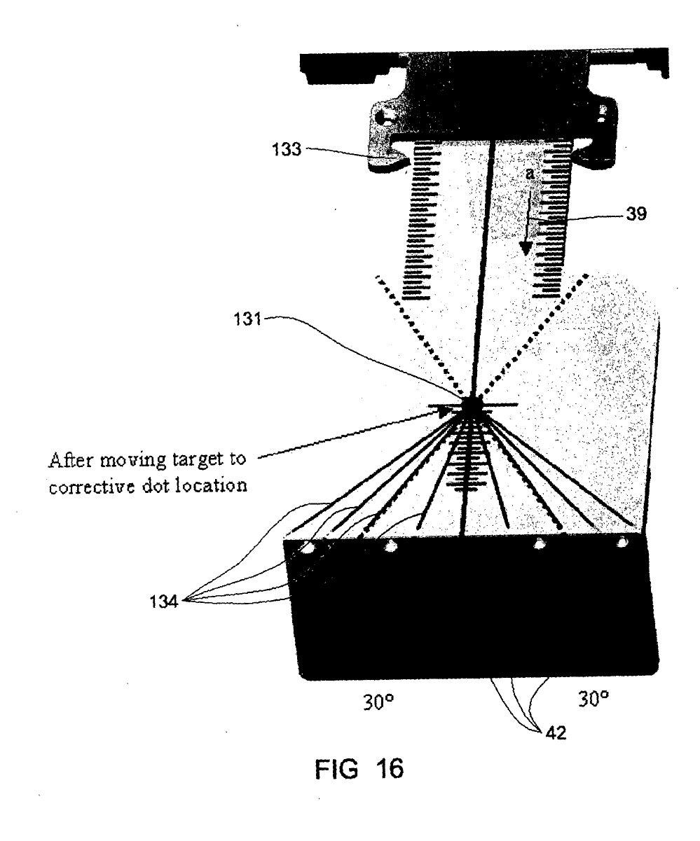

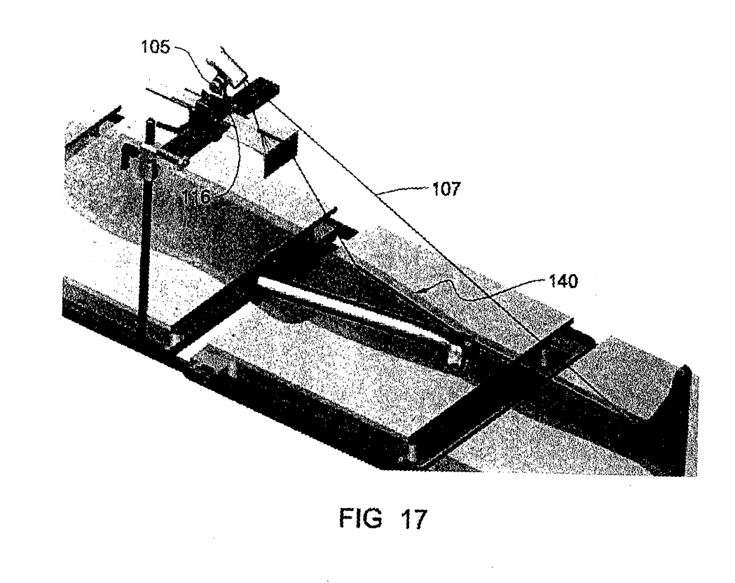

IMPORTANT: When dot L is superior to the M dot, the Knee laser adjustment is medial. If dot M is superior to the L dot, Knee laser adjustment is made laterally.
e. Turning to (FIG. 14) Carefully measure the distance between dot 120 L and dot 121 M with a ruler or by counting the number of 2 mm lines on the built-in ruler 124 results in a distance “e” 128 divide this distance “e” by two. Draw a short midline transverse axis 130 between the dots (FIG. 14).
f. Using the same midline distance in mm (one-half of “e”) make a Corrective dot 131 in the direction of the correction on this midline 130 (FIG. 14).
g. With the Knee Laser beam 107 returned to centerline 117 of the Target (FIGS. 12 and 13), dial the ADJUSTMENT KNOB 66 (FIG. 15) on the Distal Femoral Resector Guide so the knee laser beam 107 is in a parallel position 132 with the Corrective Dot 131 (FIG. 14). This adjustment will correct the longitudinal axis of the Knee to the center of the femoral head (FIGS. 14 and 16).
   SECOND: Adjust the Longitudinal and Transverse Axis of the
   Target to Knee Laser corrective dot position:
a. Move the Target transverse axis (bulls-eye) by pulling the Target longitudinally 39 to the midpoint of the Corrective Dot 131. Measure this distance from a fixed position on the Target 133 (FIG. 16).
b. Now make the Knee Laser beam 107 co-linear with the Target centerline 117 by moving the target transversely 33 (FIG. 11). There is only one position the bulls-eye will line up with the medial and lateral radial lines 134 for this patient, which is the exact center of the femoral head (FIG. 16).

For the perfectionist, minor adjustments can be made here by moving the bulls-eye longitudinally and adjusting the knee laser ADJUSTMENT KNOB to match the selected radial line. After making an adjustment knob change, be sure to always make the laser co-linear with the Target's centerline BY MOVING the Target transversely. If it is not lining up, simply repeat the FIRST STEP from your present location.

The Bulls-Eye of the Laser Target is Now Positioned Directly Over The Center of the Femoral Head, and the Beam is Showing the Leg'S Mechanical Axis.

Check the results by moving the Knee Laser beam 107 medially and laterally keeping it parallel with the vertical lines 42 on the front of the target and the radial lines 134 on the top of the target. It should transverse through the center of the Target bulls-eye 36. Do not worry if it is slightly off the center of the bulls-eye because the Laser is extremely sensitive. If the surgeon is within the bulls-eye quarter inch circle, they will be accurate within a quarter of a degree of the actual center of the Femoral Head. Spending a lot of time for perfection is not worth the effort because it is rare to make the resection cuts that accurate. Later minor adjustments can be made with the Mechanical Axis Longitudinal Laser Line 140 (FIG. 17) during the cementing process.

The knee laser is now removed and the distal femoral cut is made with the saw. Be sure to remove the cortical screws 71 holding the V-Frame 65 to the distal condyles before making this resection. The V-Frame itself can also be removed if loose. The laser is now moved to the second mount comprising the Target Laser Mount 116 (FIG. 17) to create the Laser Ruler 140. The laser beam 107 will have to be tilted down via the swivel 103 by turning locking knob 105 to shine on the entire leg creating a visual line 140 (Laser Ruler) on the leg. The remainder of the knee arthroplasty is done in the conventional manner.

Value of the Longitudinal Axis Ruler:

Since the hip laser is centered on the femoral head, the straight line this laser emits is the mechanical axis for this patient. Neutral alignment is when the laser passes through the center of the knee and the center of the ankle. Hold the leg parallel to this line to check your alignment after making all cuts with the trial components in place.

Helpful Hints:

While aligning the leg on the Mechanical Axis laser-line, watch out for external rotation errors. There is a tendency for the femur to externally rotate especially in heavier patients while resting on the operative table. About 10-15 degrees of rotation error can equal 1 degree of alignment error depending on the length of the patient's femur.

To avoid this error, the surgeon should stand at the end of the operating table with one hand under the distal femur rotating the femur to neutral and pushing the trial components together with his abdomen. He then must align the laser beam on the center of the knee and ankle. Deviation of the laser beam from the center of the knee is the alignment error from neutral (mechanical axis).

For alignment errors less than one-degree (6-7 mm), use bone cement to correct the error to neutral. For errors more than 1 degree (>7 mm) , be sure that rotation is not causing some of this error. Then check the tibial resection by re-applying the tibial resector and make sure this cut is perpendicular to the axis of the tibia. Tibial error is easily fixed by filing down the high side evenly. Femoral errors that cannot be filed will require re-applying the distal femoral cutting jigs.

As such, an invention has been disclosed in terms of a preferred embodiment thereof including apparatus and method for its practice that fulfills each and every one of the objects of the invention as set forth hereinabove, and provides a new and useful laser triangulation of the femoral head for total knee arthroplasty alignment instruments and surgical method of use, of great novelty and utility.

Of course, various changes, modifications and alterations in the teachings of the present invention may be contemplated by those skilled in the art without departing from the intended spirit and scope thereof.

As such, it is intended that the present invention only be limited by the terms of the appended claims.

1. A method of locating a center of a femoral head of a human leg of a patient having a femur, knee joint with a distal femur, tibia and femoral head, the method including the steps of:
a. mounting a laser beam generator on said knee joint;
b. shining a laser beam emanating from said generator toward a target located adjacent said femoral head;
c. aligning said laser beam with respect to said target to locate said center of said femoral head.

2. The method of claim 1, before said mounting step, including the step of locating the patient on a surgical table by:
a. placing said patient on said surgical table;
b. installing a clamp on said table;
c. attaching a target positioner on said clamp;
d. lifting said patient's thigh to a position perpendicular to said table and moving said patient so that said thigh in said perpendicular position engages said target positioner.

3. The method of claim 2, further including the steps of, after said lifting and moving steps:
a. maintaining said patient in position;
b. removing said target positioner from said clamp; and
c. installing said target on said clamp.

4. The method of claim 3, further including the step of adjusting said target with respect to said clamp in three degrees of freedom.

5. The method of claim 1, further including the step of adjusting said target with respect to said femoral head in three degrees of freedom.

6. The method of claim 1, wherein said target has a transverse arm portion generally perpendicular to a longitudinal extent of a surgical table on which said target is mounted that facilitates transverse adjustment of the target.

7. The method of claim 1, wherein said target has a longitudinal arm generally parallel to a longitudinal extent of a surgical table on which said target is mounted that facilitates longitudinal adjustment of the target.

8. The method of claim 7, wherein said longitudinal arm has a bulls-eye on a top surface thereof with lines radiating divergently from the bulls-eye that respectively connect to a plurality of parallel lines on a vertical front face of said target.

9. The method of claim 1, wherein said mounting step includes the steps of:
a. centralizing an intercondylar notch of said distal femur; and
b. creating a flat plane on an anterior femoral cortex of said distal femur.

10. The method of claim 9, further including the steps of, after said creating step:
a. mounting an anterior condyle resector guide on said flat plane; and
b. resecting top portions of anterior femoral condyles of said distal femur.

11. The method of claim 10, further including the steps of, after said resecting step:
a. mounting a frame centered on the intercondylar notch of said distal femur;
b. mounting a distal femoral resector guide on said frame; and
c. mounting said generator on said distal femoral resector guide.

12. The method of claim 8, wherein said shining step shines said laser beam onto said target.

13. The method of claim 8, wherein the laser beam is maintained parallel to the parallel lines on the vertical front face of said target to eliminate rotational error.

14. The method of claim 13, wherein said aligning step includes the step of triangulation of the laser beam with the leg held at least 15 degrees toward a midline thereof and marking a first position of said laser beam, and comparing said first position on said target with a position of said laser beam on said target with said leg held at least 15 degrees away from said midline.

15. The method of claim 14, wherein distance is measured between said first and second portions of said laser beam and half this distance being used to create a corrective transverse axis and longitudinal axis in an opposite direction of a more cephalad laser beam.

16. The method of claim 15, wherein the laser beam is adjusted at the knee joint to a location of corrective axes on said target.

17. The method of claim 16, wherein, thereafter, said target is adjusted both longitudinally and transversely with respect to said surgical table to be parallel to the laser beam's longitudinal axis at the knee joint.

18. The method of claim 1, after said aligning step, further including the steps of:
a. providing a laser beam generator mount on said target;
b. removing said generator from said knee joint; and
c. attaching said generator to said target mount.

19. The method of claim 18, after said attaching step, actuating said laser beam toward said distal femur along a mechanical axis of said leg.

20. A system for locating a center of a femoral head of a patient, said patient having a leg with a femoral head having a center, a femur, distal femur, knee joint and tibia, said system comprising:
a. a frame attachable to a center of a distal femoral intercondylar notch of said distal femur;
b. a laser beam generator mountable on an adjustable distal femoral resector pivotable on a frame mounted at said distal femur of said patient;
c. a target mountable over said femoral head;
d. whereby with said generator mounted on said distal femur and said target mounted adjacent said femoral head, a laser beam from said generator may be shined toward said target, and position of said target and generator may be adjusted to align said laser beam over the center of said femoral head.

21. The system of claim 20, wherein said frame has a wing structure matching anatomy of the distal femoral intercondylar notch which includes diverging portions that typically diverge between 38-42 degrees.

22. The system of claim 21, wherein the frame has a post at a center thereof about which said attachable distal femoral resector may pivot.

23. The system of claim 22, wherein the distal femoral resector is adjustable through movements of an adjustment knob attached to said frame.

24. The system of claim 20, wherein said distal femoral resector has a tapered mounting slot receiving said generator that prevents wobble of said laser beam.

25. The system of claim 20, wherein said distal femoral resector and frame have interrelated indicia permitting determination of relative position of said frame with respect to the said resector.

26. The system of claim 20, wherein said target includes adjusting means for adjusting target location.

27. The system of claim 26, wherein said adjusting means is movable to adjust location of said target in three degrees of freedom.

28. The system of claim 27, wherein said target includes a transverse arm and a longitudinal arm generally perpendicular to said transverse arm.

29. The system of claim 28, wherein said transverse arm extends generally perpendicular to a length of said patient.

30. The system of claim 28, wherein said longitudinal arm includes a bulls-eye on a top surface thereof with divergent lines radiating from the bulls-eye that connect, respectively, to a plurality of vertical parallel lines on a front vertically extending face of said target.

31. The system of claim 20, wherein said target is mounted on a bracket fixed to a surgical table.

32. The system of claim 30, wherein said target is mounted on a bracket fixed to a surgical table.

33. The system of claim 32, wherein said top surface of said target is generally parallel to a top surface of said surgical table.

34. The system of claim 20, wherein said generator is mountable on said distal femur with a first mount, and further including a second mount for mounting said generator on said target.

35. The system of claim 20, wherein said laser beam generator comprises a canister with an internal chamber, a removable cover adapted to close said chamber and a laser beam source removably insertable into said chamber.

36. The system of claim 35, wherein said canister has a bracket with a tapered shoe attached thereto for mounting said generator.

37. The system of claim 36, wherein said canister has a wall opposite said cover, said wall having a lens aligned with said laser beam when said laser beam source is within said chamber.

38. The system of claim 37, further including alignment means for aligning said laser beam source in said chamber.

39. The system of claim 35, wherein said laser beam source has an outer surface on which an on-off switch is located, said switch engaging a cooperating surface within said chamber when said laser beam source is inserted into said chamber to activate said laser beam.

40. The system of claim 36, wherein said tapered shoe permits mounting said generator on said distal femoral resector or on said target.

41. A system for locating a center of a femoral head of a patient, said patient having a leg with a femoral head having a center, a femur, distal femur, knee joint and tibia, said system comprising:
a) a frame attachable to said distal femur;
b) a light beam generator mountable on said frame;
c) a target mountable over said femoral head;
d) whereby, with said generator mounted on said frame and said target mounted adjacent said femoral head, a light beam from said generator may be shined toward said target, and position of said target and generator may be adjusted to align said light beam over the center of said femoral head.
